(19) United States
(12) Patent Application Publication
(10) Pub. No.: US 2021/0085267 A1
(43) Pub. Date: Mar. 25, 2021

(54) RADIOGRAPHIC IMAGE PROCESSING APPARATUS, RADIOGRAPHIC IMAGE PROCESSING METHOD, AND RADIOGRAPHIC IMAGE PROCESSING PROGRAM

(71) Applicant: FUJIFILM CORPORATION, Tokyo (JP)

(72) Inventor: Shin HAMAUZU, Kanagawa (JP)

(21) Appl. No.: 17/002,793

(22) Filed: Aug. 26, 2020

(30) Foreign Application Priority Data
Sep. 25, 2019 (JP) 2019-173855

(51) Int. Cl. A61B 6/12; A61B 6/00; A61F 13/44; G06T 7/00; G06T 7/70

(52) U.S. Cl. CPC A61B 6/12; A61B 6/463; A61F 13/44; G06T 2207/30004; G06T 7/70; G06T 2207/10116; G06T 7/0012

(57) ABSTRACT

A first detection unit detects a region of a first surgical tool which should not be present in a body of a patient from a radiographic image of the patient. A second detection unit detects a region of a second surgical tool which should be or may be present in the body of the patient from the radiographic image. In a case in which at least one of the region of the first surgical tool or the region of the second surgical tool is detected, a display control unit displays the radiographic image on a display unit such that the detected region of the surgical tool is highlighted.

### CROSS REFERENCE TO RELATED APPLICATIONS

The present application claims priority under 35 U.S.C. § 119 to Japanese Patent Application No. 2019-173855 filed on Sep. 25, 2019. The above application is hereby expressly incorporated by reference, in its entirety, into the present application.

### BACKGROUND OF THE INVENTION

### Technical Field

The present disclosure relates to a radiographic image processing apparatus, a radiographic image processing method, and a radiographic image processing program.

### Related Art

Various surgical tools, such as gauze for suppressing bleeding, a thread and a needle for sewing up a wound, a scalpel and scissors for incision, a drain for draining blood, and forceps for opening incision, are used in a case in which a surgical operation is performed for a patient. The surgical tools may cause serious complications in a case in which they remain in the body of the patient after surgery. Therefore, it is necessary to check that no surgical tools remain in the body of the patient after surgery. However, there is a possibility that the remaining surgical tool will be overlooked by visual confirmation in an exhausted state after surgery.

Therefore, a method has been proposed which detects a foreign material in an image of a patient and displays the foreign material so as to be highlighted. For example, JP2017-202310A discloses a method that, in a case in which a foreign material, such as metal, is detected in a CT image of a patient, presents the detected foreign material on the image. Further, JP2018-517950A discloses a method that acquires a video image of a surgical site with a camera and displays a non-tissue region, such as gauze, in the video image so as to be highlighted.

However, CT images are not capable of being captured in an operating room and it is necessary to move a patient to an imaging room for CT imaging after surgery. Therefore, even in a case in which a foreign material is found in the body of the patient by the method described in JP2017-202310A, surgery needs to be performed again in order to remove the foreign material, which imposes a heavy burden on the patient. In addition, gauze is used to be pushed into the body of the patient and is stained with blood. Therefore, it is difficult to find gauze using the video image acquired by the camera as in the method described in JP2018-517950A. Further, in many cases, the surgical field is blocked by the operator. Therefore, it is difficult to always ensure the surgical field in order to acquire a video image. For this reason, there is a possibility that a surgical tool will remain in the body of the patient after the surgery in the method described in JP2018-517950A. Further, it is very rare that a surgical tool remains in the body. Therefore, in a case in which the surgical tool is not detected, it is difficult for the operator to determine whether or not a detection process is operating correctly.

### SUMMARY OF THE INVENTION

The present disclosure has been made in view of the above-mentioned problems and an object of the present disclosure is to provide a technique which can check whether or not a surgical tool detection process is operating correctly.

According to the present disclosure, there is provided a radiographic image processing apparatus comprising: a first detection unit that detects a region of a first surgical tool which should not be present in a body of a patient from a radiographic image of the patient; a second detection unit that detects a region of a second surgical tool which should be or may be present in the body of the patient from the radiographic image; and a display control unit that, in a case in which at least one of the region of the first surgical tool or the region of the second surgical tool is detected, displays the radiographic image on a display unit such that the detected region of the surgical tool is highlighted.

The “radiographic image” in the present disclosure means a two-dimensional image which is a fluoroscopic image of a subject acquired by irradiating the subject with radiation. The radiographic image may be a still image or a moving image. For example, the radiographic image may be acquired by a portable radiation detector. In some cases, the operator performs surgery while observing the radiographic image of the patient using a C-arm fluoroscopic apparatus. In this case, the radiographic image may be acquired by the C-arm fluoroscopic apparatus.

In the radiographic image processing apparatus according to the present disclosure, in a case in which both the region of the first surgical tool and the region of the second surgical tool are detected, the display control unit may display the radiographic image on the display unit such that at least one of the region of the first surgical tool or the region of the second surgical tool is highlighted.

In the radiographic image processing apparatus according to the present disclosure, the display control unit may display the radiographic image on the display unit such that at least the region of the first surgical tool is highlighted.

In the radiographic image processing apparatus according to the present disclosure, in a case in which the region of the first surgical tool and the region of the second surgical tool are not detected, the display control unit may notify the fact.

In the radiographic image processing apparatus according to the present disclosure, the radiographic image may be acquired by a portable radiation detector or an imaging apparatus that is installed in an operating room for performing surgery on the patient.

In the radiographic image processing apparatus according to the present disclosure, the first detection unit may include a discriminator trained so as to discriminate the region of the first surgical tool in an input radiographic image.

In the radiographic image processing apparatus according to the present disclosure, the second detection unit may include a discriminator trained so as to discriminate the region of the second surgical tool in an input radiographic image.

In the radiographic image processing apparatus according to the present disclosure, the first surgical tool may include at least one of gauze, a scalpel, scissors, a drain, a needle, a thread, or forceps.

In this case, at least a portion of the gauze may include a radiation absorbing thread.

In the radiographic image processing apparatus according to the present disclosure, the second surgical tool may include at least one of a stent, a pacemaker, a ureteral catheter, a rod, or a screw.

According to the present disclosure, there is provided a radiographic image processing method comprising: detecting a region of a first surgical tool which should not be present in a body of a patient from a radiographic image of the patient; detecting a region of a second surgical tool which should be or may be present in the body of the patient from the radiographic image; and in a case in which at least one of the region of the first surgical tool or the region of the second surgical tool is detected, displaying the radiographic image on a display unit such that the detected region of the surgical tool is highlighted.

In addition, a program that causes a computer to perform the radiographic image processing method according to the present disclosure may be provided.

Another radiographic image processing apparatus according to the present disclosure comprises a memory that stores commands to be executed by a computer and a processor that is configured to execute the stored commands. The processor performs: a process of detecting a region of a first surgical tool which should not be present in a body of a patient from a radiographic image of the patient; a process of detecting a region of a second surgical tool which should be or may be present in the body of the patient from the radiographic image; and a process of, in a case in which at least one of the region of the first surgical tool or the region of the second surgical tool is detected, displaying the radiographic image on a display unit such that the detected region of the surgical tool is highlighted.

According to the present disclosure, it is possible to check whether the surgical tool detection process is operating correctly.

### BRIEF DESCRIPTION OF THE DRAWINGS

FIG. 1 is a block diagram schematically illustrating a configuration of a radiography system to which a radiographic image processing apparatus according to an embodiment of the present disclosure is applied.

FIG. 2 is a diagram schematically illustrating a configuration of the radiographic image processing apparatus according to this embodiment.

FIG. 3 is a diagram illustrating gauze.

FIG. 4 is a diagram illustrating a radiographic image including a first surgical tool.

FIG. 5 is a diagram illustrating a display screen for a radiographic image in which the first surgical tool is highlighted.

FIG. 6 is a diagram illustrating a display screen for a radiographic image in which a second surgical tool is highlighted.

FIG. 7 is a diagram illustrating a display screen for a radiographic image in which the first surgical tool is highlighted in a case in which both the first surgical tool and the second surgical tool are detected.

FIG. 8 is a diagram illustrating a display screen for a radiographic image in which the first surgical tool is highlighted.

FIG. 9 is a diagram illustrating a notification screen in a case in which a surgical tool is not detected.

FIG. 10 is a flowchart illustrating a process performed in this embodiment.

### DETAILED DESCRIPTION

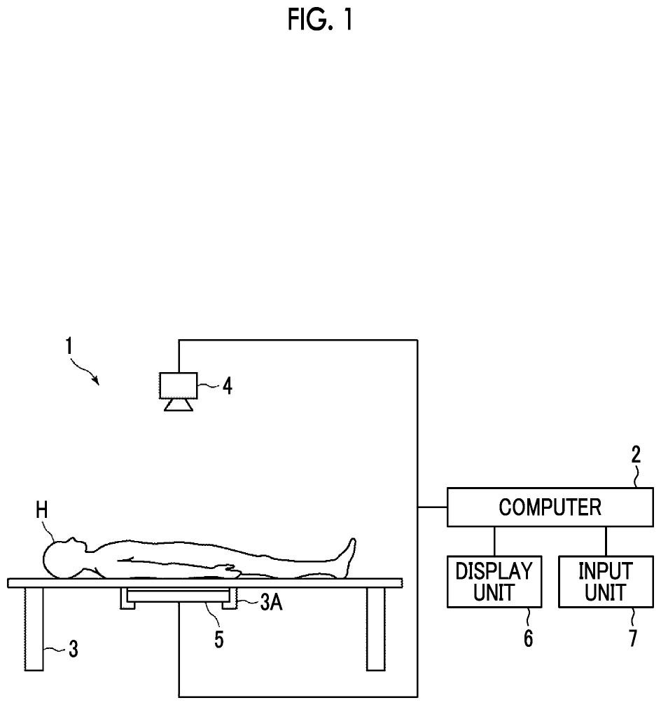

Hereinafter, embodiments of the present disclosure will be described with reference to the drawings. FIG. 1 is a block diagram schematically illustrating a configuration of a radiography system to which a radiographic image processing apparatus according to an embodiment of the present disclosure is applied. As illustrated in FIG. 1, the radiography system according to this embodiment acquires a radiographic image of a subject as a patient after a surgical operation and detects a surgical tool included in the radiographic image. The radiography system according to this embodiment includes an imaging apparatus 1 and a computer 2 which is the radiographic image processing apparatus according to this embodiment.

The imaging apparatus 1 detect radiation, which has been emitted from a radiation source 4, such as an X-ray source, with a radiation detector 5, and transmitted through a subject H to acquire a radiographic image G0 of the subject H that lies supine on an operating table 3. The radiographic image G0 is input to the computer 2 which is the radiographic image processing apparatus.

The radiation detector 5 can repeatedly perform the recording and reading of a radiographic image and may be a so-called direct-type radiation detector that directly receives the emitted radiation and generates charge or a so-called indirect-type radiation detector that converts radiation into visible light and then converts the visible light into a charge signal. As a method for reading a radiographic image signal, it is desirable to use a so-called thin film transistor (TFT) reading method which turns on and off a TFT switch to read a radiographic image signal or a so-called optical reading method which emits reading light to read a radiographic image signal. However, the present disclosure is not limited thereto and other methods may be used.

The radiation detector 5 is a portable radiation detector and is attached to the operating table 3 by an attachment portion 3A that is provided in the operating table 3. The radiation detector 5 may be fixed to the operating table 3.

A display unit 6 and an input unit 7 are connected to the computer 2. The display unit 6 consists of a display, for example, a cathode ray tube (CRT) or a liquid crystal display and assists the input of a radiographic image acquired by imaging and various kinds of data necessary for processes performed by the computer 2. The input unit 7 consists of, for example, a keyboard, a mouse, or a touch panel.

A radiographic image processing program according to this embodiment is installed in the computer 2. In this embodiment, the computer 2 may be a workstation or a personal computer that is directly operated by an operator or a server computer that is connected to the computer through a network. The radiographic image processing program is stored in a storage device of the server computer connected to the network or a network storage so as to be accessed from the outside and is downloaded and installed in the computer on demand. Alternatively, the radiographic image processing program is recorded on a recording medium such as a digital versatile disc (DVD) or a compact disc read only memory (CD-ROM), is distributed, and is installed in the computer from the recording medium.

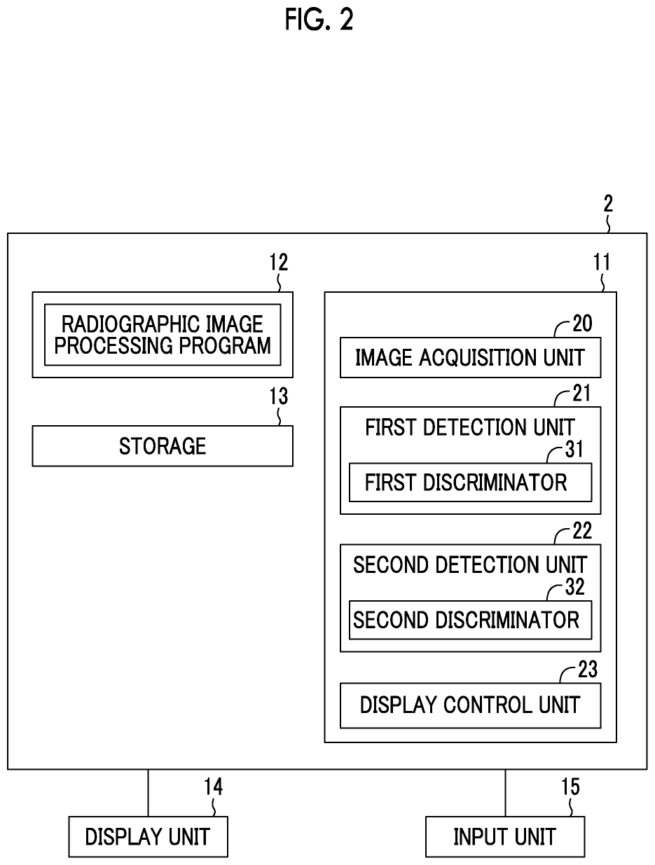

FIG. 2 is a diagram schematically illustrating the configuration of the radiographic image processing apparatus implemented by installing, for example, the radiographic image processing program in the computer 2 in this embodiment. As illustrated in FIG. 2, the radiographic image processing apparatus includes a central processing unit (CPU) 11, a memory 12, and a storage 13 as a standard computer configuration.

The storage 13 consists of a storage device, such as a hard disk or a solid state drive (SSD), and stores various kinds of information including an imaging program for driving each unit of the imaging apparatus 1 and the radiographic image processing program. In addition, the radiographic image acquired by imaging is stored in the storage 13.

The memory 12 temporarily stores, for example, the radiographic image processing program stored in the storage 13 in order to cause the CPU 11 to perform various processes. The radiographic image processing program defines the following processes as the processes to be performed by the CPU 11: an image acquisition process that detect radiation with a radiation detector 5 detect radiation, which has been emitted from the radiation source 4, with a radiation detector 5, and then transmitted through the subject H, to acquire the radiographic image G0; a first detection process that detects a region of a first surgical tool in the radiographic image; a second detection process that detects a region of a second surgical tool in the radiographic image; and a display control process that, in a case in which at least one of the first surgical tool or the second surgical tool is detected, displays the radiographic image on the display unit 6 such that the detected region of the surgical tool is highlighted.

The CPU 11 performs the above-mentioned processes according to the radiographic image processing program such that the computer 2 functions as an image acquisition unit 20, a first detection unit 21, a second detection unit 22, and a display control unit 23.

The first surgical tool is a surgical tool that should not be present in the body of the patient after surgery. Examples of the first surgical tool include gauze, a scalpel, scissors, a drain, a needle, a thread, and forceps. The second surgical tool is a surgical tool that should or may be present in the body of the patient after surgery. Examples of the second surgical tool include a stent, a pacemaker, a ureteral catheter, a rod, and a screw. The rod is a rod-shaped metal part for connecting fractured bones. The screw is a metal part that attaches the rod to the bones.

The image acquisition unit 20 drives the radiation source 4 to irradiate the subject H that has undergone surgery with radiation and detects the radiation transmitted through the subject H using the radiation detector 5 to acquire the radiographic image G0. In this case, the image acquisition unit 20 sets imaging conditions, such as the type of target and filter used in the radiation source 4, an imaging dose, a tube voltage, and a SID.

The first detection unit 21 detects the region of the first surgical tool in the radiographic image G0. For the detection, a first discriminator 31 that discriminates the region of the first surgical tool included in the radiographic image G0 in a case in which the radiographic image G0 is input is applied to the first detection unit 21. Therefore, in a case in which the target radiographic image G0 is input to the first detection unit 21, the first detection unit 21 directs the first discriminator 31 to extract the region of the first surgical tool included in the radiographic image G0, thereby detecting the region of the first surgical tool.

Here, the first discriminator 31 is constructed by training a machine learning model using the radiographic image including the first surgical tool as training data. In this embodiment, it is assumed that gauze is used as the first surgical tool.

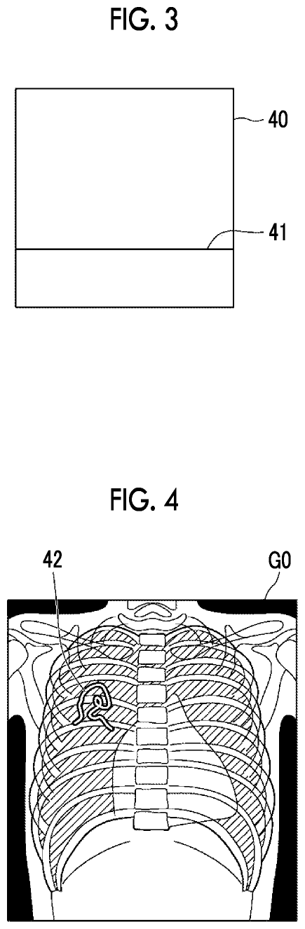

FIG. 3 is a diagram illustrating gauze. As illustrated in FIG. 3, gauze 40 is a plain-woven cotton fabric and a radiation absorbing thread 41 is woven in a portion of the gauze 40. Cotton yarn transmits radiation and the radiation absorbing thread 41 absorbs radiation. Therefore, the radiographic image of the gauze 40 includes only the linear radiation absorbing thread 41. Here, during surgery, the gauze 40 is rolled and inserted into the human body in order to absorb blood. Therefore, in a case in which the gauze 40 is present in the human body, as illustrated in FIG. 4, a gauze image 42 which is the image of the gauze 40 included in the radiographic image G0 represents a state in which the radiation absorbing thread 41 is curled.

The second detection unit 22 detects the region of the second surgical tool in the radiographic image G0. For the detection, a second discriminator 32 that discriminates the region of the second surgical tool included in the radiographic image G0 in a case in which the radiographic image G0 is input is applied to the second detection unit 22. Therefore, in a case in which the target radiographic image G0 is input to the second detection unit 22, the second detection unit 22 directs the second discriminator 32 to extract the region of the second surgical tool included in the radiographic image G0, thereby detecting the region of the second surgical tool.

Here, the second discriminator 32 is constructed by training the machine learning model using the radiographic image including the second surgical tool as training data. In this embodiment, it is assumed that the second surgical tool is a stent that is inserted into the coronary artery of the heart.

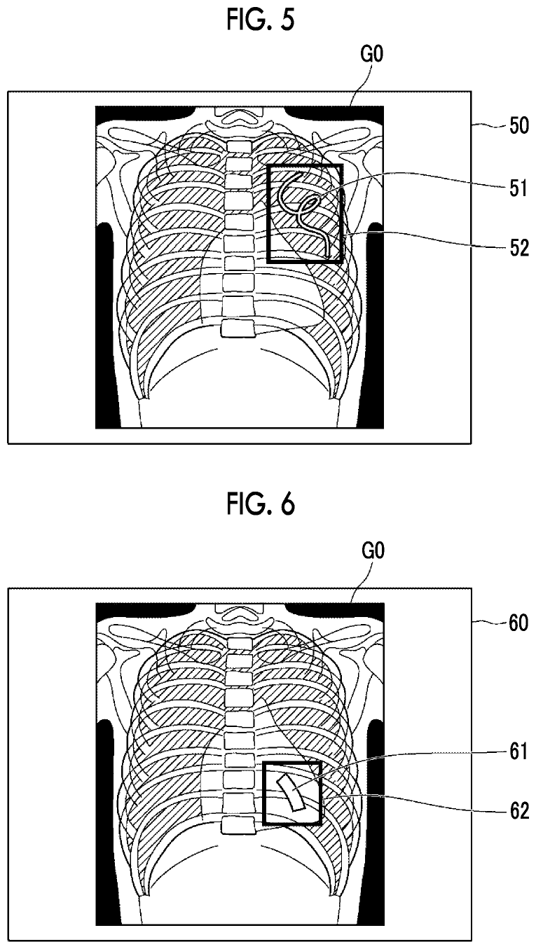

In a case in which at least one of the region of the first surgical tool or the region of the second surgical tool is detected, the display control unit 23 displays the radiographic image G0 on the display unit 6 such that the detected region of the surgical tool is highlighted. Specifically, in a case in which only the first surgical tool is detected, the display control unit 23 displays the radiographic image G0 on the display unit 6 such that the region of the first surgical tool is highlighted. FIG. 5 is a diagram illustrating a display screen for a radiographic image in which the region of the first surgical tool is highlighted. As illustrated in FIG. 5, the radiographic image G0 is displayed on a display screen 50. In the radiographic image G0, a region 51 of the gauze as the first surgical tool is detected. The region 51 of the gauze is highlighted by being surrounded by a frame 52.

On the other hand, in a case in which only the second surgical tool is detected, the display control unit 23 displays the radiographic image G0 on the display unit 6 such that the region of the second surgical tool is highlighted. FIG. 6 is a diagram illustrating a display screen for a radiographic image in which the region of the second surgical tool is highlighted. As illustrated in FIG. 6, the radiographic image G0 is displayed on a display screen 60. In the radiographic image G0, a region 61 of the stent as the second surgical tool is detected. The region 61 of the stent is highlighted by being surrounded by a frame 62.

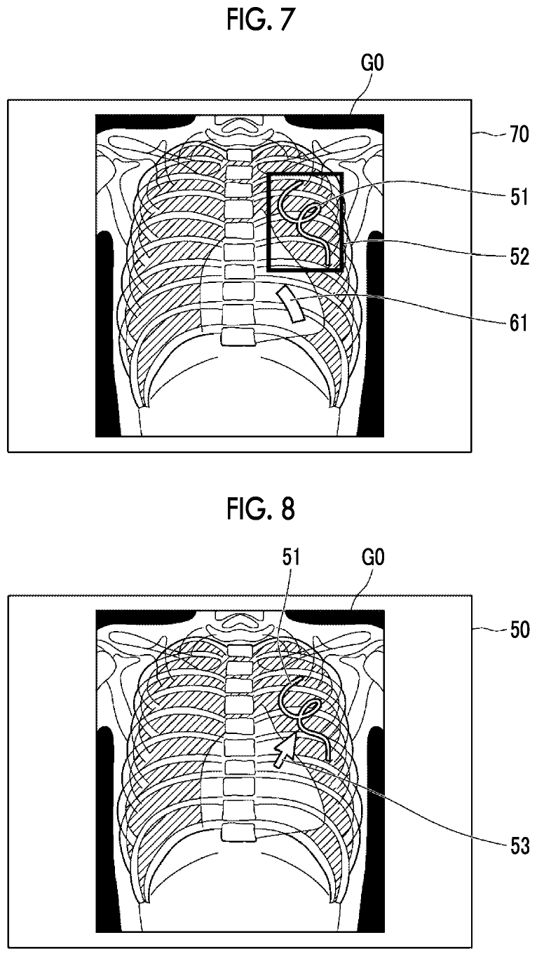

Further, in a case in which both the region of the first surgical tool and the region of the second surgical tool are detected, the display control unit 23 displays the radiographic image G0 on the display unit 6 such that only the region of the first surgical tool is highlighted. FIG. 7 is a diagram illustrating a display screen for a radiographic image in which only the region of the first surgical tool is highlighted. As illustrated in FIG. 7, the radiographic image G0 is displayed on a display screen 70. In the radiographic image G0, the region 51 of the gauze as the first surgical tool and the region 61 of the stent as the second surgical tool are detected. Only the region 51 of the gauze is highlighted by being surrounded by the frame 52.

In addition, in a case in which the region of the surgical tool is highlighted, for example, a color may be added to the detected region of the surgical tool, instead of adding the frame. As illustrated in FIG. 8, a mark 53, such as an arrow or an asterisk, may be added in the vicinity of the region 51 of the first surgical tool to highlight the detected region of the first surgical tool.

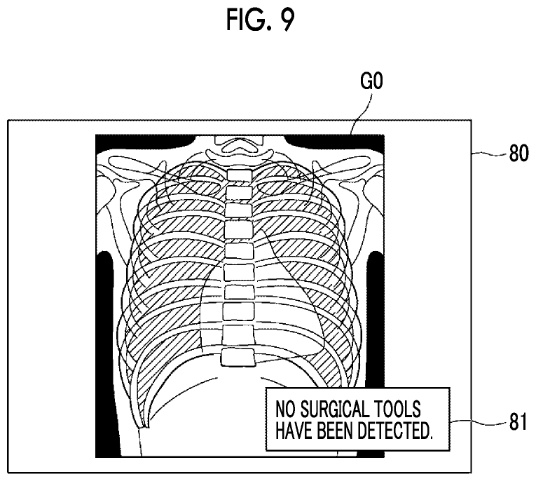

In a case in which neither the region of the first surgical tool nor the region of the second surgical tool is detected, the display control unit 23 notifies the fact. FIG. 9 is a diagram illustrating a notification screen in a case in which the region of the first surgical tool and the region of the second surgical tool are not detected. As illustrated in FIG. 9, a message 81 of “No surgical tools have been detected.” is displayed on a display screen 80 so as to be superimposed on the radiographic image G0.

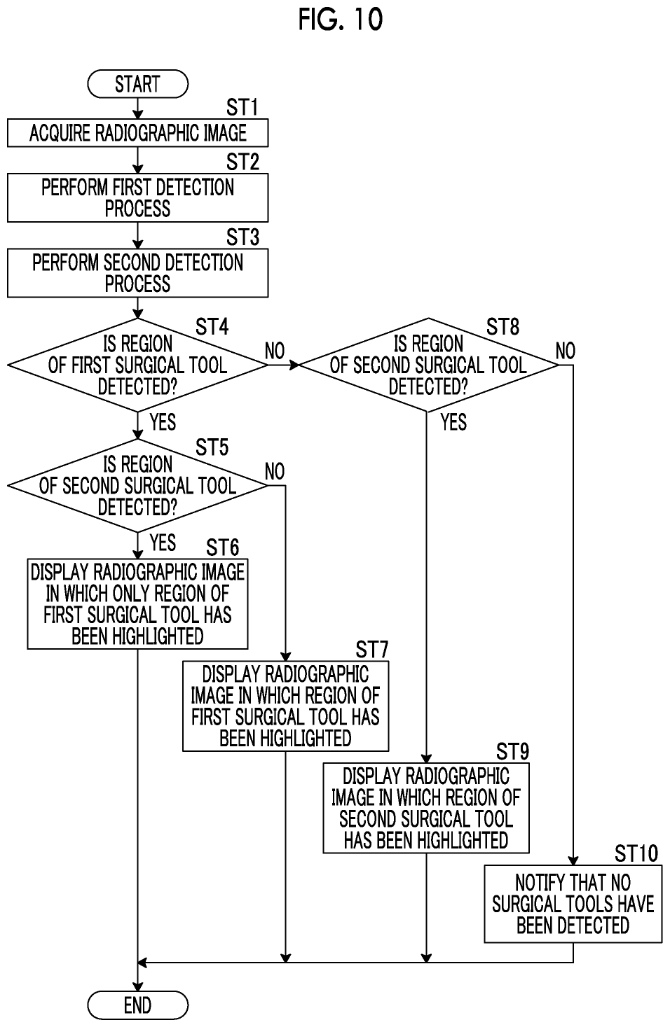

Next, a process performed in this embodiment will be described. FIG. 10 is a flowchart illustrating the process performed in this embodiment. The image acquisition unit 20 acquires the radiographic image G0 as a detection target (Step ST1) and the first detection unit 21 performs a first detection process of detecting the region of the first surgical tool from the radiographic image G0 (Step ST2). In addition, the second detection unit 22 performs a second detection process of detecting the region of the second surgical tool from the radiographic image G0 (Step ST3).

Then, the display control unit 23 determines whether or not the region of the first surgical tool has been detected from the radiographic image G0 (Step ST4). In a case in which the determination result in Step ST4 is “Yes”, the display control unit 23 determines whether or not the region of the second surgical tool has been detected from the radiographic image G0 (Step ST5). In a case in which the determination result in Step ST5 is “Yes”, the display control unit 23 displays the radiographic image G0 in which only the region of the first surgical tool has been highlighted on the display unit 6 since both the region of the first surgical tool and the region of the second surgical tool are detected from the radiographic image G0 (Step ST6). Then, the process ends. In a case in which the determination result in Step ST5 is “No”, the display control unit 23 displays the radiographic image G0 in which the region of the first surgical tool has been highlighted on the display unit 6 since only the region of the first surgical tool is detected from the radiographic image G0 (Step ST7). Then, the process ends.

On the other hand, in a case in which the determination result in Step ST4 is “No”, the display control unit 23 determines whether or not the region of the second surgical tool has been detected from the radiographic image G0 (Step ST8). In a case in which the determination result in Step ST8 is “Yes”, the display control unit 23 displays the radiographic image G0 in which only the region of the second surgical tool has been highlighted on the display unit 6 since only the region of the second surgical tool is detected from the radiographic image G0 (Step ST9). Then, the process ends. In a case in which the determination result in Step ST8 is “No”, the display control unit 23 notifies that the region of the surgical tool has not been detected since both the region of the first surgical tool and the region of the second surgical tool are not detected from the radiographic image G0 (Step ST10). Then, the process ends.

As described above, in this embodiment, in a case in which at least one of the region of the first surgical tool or the region of the second surgical tool is detected in the radiographic image G0 of the patient, the radiographic image G0 is displayed such that the detected region of the surgical tool is highlighted.

Here, the surgical tools include the first surgical tool that should not be present in the body of the patient after surgery and the second surgical tool that should be or may be present in the body of the patient. In a case in which surgery for implanting or attaching the second surgical tool into the body of the patient is performed, only the region of the second surgical tool should be detected. According to this embodiment, the region of the second surgical tool is detected. In a case in which only the region of the second surgical tool is detected, the region of the second surgical tool is displayed so as to be highlighted.

Therefore, in a case in which the detection process operates correctly and the region of the second surgical tool is detected, the radiographic image G0 in which the region of the second surgical tool has been highlighted is displayed. On the other hand, in a case in which the detection process does not operate correctly, the region of the second surgical tool is not detected. Therefore, the region of the second surgical tool is not displayed so as to be highlighted. Therefore, the operator can see the displayed radiographic image G0 and easily check whether or not the detection process is operating correctly on the basis of whether or not the region of the second surgical tool is highlighted in the radiographic image G0.

In a case in which the detection process does not operate correctly, for example, the radiographic image G0 may be displayed on a display device with a larger screen such that the operator can check in more detail whether or not the first surgical tool is present.

Further, in this embodiment, in a case in which the region of the surgical tool is not detected in the radiographic image G0, the fact is notified. Therefore, the operator can more easily check whether or not the region of the second surgical tool is detected in the radiographic image G0.

In the above-described embodiment, the first detection unit 21 and the second detection unit 22 detect the first and second surgical tools using the first discriminator 31 and the second discriminator 32, respectively. However, the present disclosure is not limited thereto. A histogram of the radiographic image G0 may be calculated and it may be determined whether or not the histogram includes signal values of the first and second surgical tools to detect the regions of the first and second surgical tools from the radiographic image G0.

Further, in the above-described embodiment, in a case in which both the region of the first surgical tool and the region of the second surgical tool have been detected, the display control unit 23 displays the radiographic image G0 on the display unit 6 such that only the region of the first surgical tool is highlighted. However, the display control unit 23 may display the radiographic image G0 on the display unit 6 such that only the region of the second surgical tool is highlighted. In addition, both the region of the first surgical tool and the region of the second surgical tool may be highlighted.

Further, in the above-described embodiment, the radiographic image G0 is a still image. However, the present disclosure is not limited thereto. The radiographic image G0 may be a moving image. In this case, the process of detecting the regions of the first surgical tool and the second surgical tool is performed on each frame of the radiographic image G0 which is a moving image.

Further, in the above-described embodiment, the radiographic image G0 of the subject H is acquired by the radiation source 4 and the radiation detector 5. However, the present disclosure is not limited thereto. For example, in some cases, the operator performs surgery while observing the radiographic image G0 of the patient using a C-arm fluoroscopic apparatus. In this case, the radiographic image G0 may be acquired by the C-arm fluoroscopic apparatus.

Further, in the above-described embodiment, in some cases, the radiographic image G0 in which at least one of the region of the first surgical tool or the region of the second surgical tool has been detected is transmitted to an external apparatus such as an image storage server. In this case, information indicating that the region of the surgical tool has been detected may be given to the radiographic image G0. For example, in a protocol such as digital imaging and communication in medicine (DICOM) that defines the storage format of image data and communication between apparatuses, information can be given to the radiographic image G0. Therefore, the DICOM protocol may be used to give information indicating that at least one of the region of the first surgical tool or the region of the second surgical tool has been detected to the radiographic image G0.

In the above-described embodiment, the gauze 40 as the first surgical tool is the target of the first detection process. However, the present disclosure is not limited thereto. Any surgical tools, such as a scalpel, scissors, a drain, a needle, a thread, and forceps, that are used during surgery and should not be present in the body of the patient after the surgery may be used as the first surgical tool and may be the target of the first detection process. Further, the first discriminator 31 may be trained so as to discriminate the target first surgical tool. In addition, the first discriminator 31 is trained so as to detect a plurality of channels, which makes it possible to construct the first discriminator 31 so as to discriminate not only one kind of first surgical tool but also a plurality of kinds of first surgical tools.

In the above-described embodiment, the stent as the second surgical tool is the target of the second detection process. However, the present disclosure is not limited thereto. Any surgical tools, such as a pacemaker, a ureteral catheter, a rod, and a screw, that are used during surgery and should be or may be present in the body of the patient after the surgery may be used as the second surgical tool and may be the target of the second detection process. Further, the second discriminator 32 may be trained so as to discriminate the target second surgical tool. The second discriminator 32 is trained so as to detect a plurality of channels, which makes it possible to construct the second discriminator 32 so as to discriminate not only one kind of second surgical tool but also a plurality of kinds of second surgical tools.

In addition, in the above-described embodiment, the radiation is not particularly limited. For example, α-rays and γ-rays other than X-rays can be applied.

In the above-described embodiment, for example, the following various processors can be used as a hardware structure of processing units performing various processes, such as the image acquisition unit 20, the first detection unit 21, the second detection unit 22, and the display control unit 23. The various processors include, for example, a CPU which is a general-purpose processor executing software (program) to function as various processing units, a programmable logic device (PLD), such as a field programmable gate array (FPGA), which is a processor whose circuit configuration can be changed after manufacture, and a dedicated electric circuit, such as an application-specific integrated circuit (ASIC), which is a processor having a dedicated circuit configuration designed to perform a specific process.

One processing unit may be configured by one of the various processors or a combination of two or more processors of the same type or different types (for example, a combination of a plurality of FPGAs or a combination of a CPU and an FPGA). Further, a plurality of processing units may be configured by one processor.

A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As such, various processing units are configured by using one or more of the various processors as the hardware structure.

Furthermore, specifically, an electric circuit (circuitry) obtained by combining circuit elements, such as semiconductor elements, can be used as the hardware structure of the various processors.

What is claimed is:

1. A radiographic image processing apparatus comprising at least one processor, wherein the processor is configured to:
detect a region of a first surgical tool which should not be present in a body of a patient from a radiographic image of the patient;
detect a region of a second surgical tool which should be or may be present in the body of the patient from the radiographic image; and
in a case in which at least one of the region of the first surgical tool or the region of the second surgical tool is detected, display the radiographic image on a display such that the detected region of the surgical tool is highlighted.

2. The radiographic image processing apparatus according to claim 1,
wherein, in a case in which both the region of the first surgical tool and the region of the second surgical tool are detected, the processor is configured to display the radiographic image on the display such that at least one of the region of the first surgical tool or the region of the second surgical tool is highlighted.

3. The radiographic image processing apparatus according to claim 2,
wherein the processor is configured to display the radiographic image on the display such that at least the region of the first surgical tool is highlighted.

4. The radiographic image processing apparatus according to claim 1,
wherein, in a case in which the region of the first surgical tool and the region of the second surgical tool are not detected, the processor is configured to notify the fact.

5. The radiographic image processing apparatus according to claim 1,
wherein the radiographic image is acquired by a portable radiation detector or an imaging apparatus that is installed in an operating room for performing surgery on the patient.

6. The radiographic image processing apparatus according to claim 1,
wherein the processor is configured to discriminate the region of the first surgical tool in the radiographic image of the patient by using a discriminator trained so as to discriminate the region of the first surgical tool in an input radiographic image.

7. The radiographic image processing apparatus according to claim 1,
wherein the processor is configured to discriminate the region of the second surgical tool in the radiographic image of the patient by using a discriminator trained so as to discriminate the region of the second surgical tool in an input radiographic image.

8. The radiographic image processing apparatus according to claim 1,
wherein the first surgical tool includes at least one of gauze, a scalpel, scissors, a drain, a needle, a thread, or forceps.

9. The radiographic image processing apparatus according to claim 8,
wherein at least a portion of the gauze includes a radiation absorbing thread.

10. The radiographic image processing apparatus according to claim 1,
wherein the second surgical tool includes at least one of a stent, a pacemaker, a ureteral catheter, a rod, or a screw.

11. A radiographic image processing method comprising:
detecting a region of a first surgical tool which should not be present in a body of a patient from a radiographic image of the patient;
detecting a region of a second surgical tool which should be or may be present in the body of the patient from the radiographic image; and
in a case in which at least one of the region of the first surgical tool or the region of the second surgical tool is detected, displaying the radiographic image on a display such that the detected region of the surgical tool is highlighted.

12. A non-transitory computer-readable storage medium that stores a radiographic image processing program that causes a computer to perform:
detecting a region of a first surgical tool which should not be present in a body of a patient from a radiographic image of the patient;
detecting a region of a second surgical tool which should be or may be present in the body of the patient from the radiographic image; and
in a case in which at least one of the region of the first surgical tool or the region of the second surgical tool is detected, displaying the radiographic image on a display such that the detected region of the surgical tool is highlighted.
